(19) United States
(12) Patent Application Publication
(10) Pub. No.: US 2004/0109831 A1
(43) Pub. Date: Jun. 10, 2004

(54) Cosmetic compositions for the protection and optical enhancement of tattooed skin

(75) Inventor: Richard C. Dodwell, Austin, TX (US)

Correspondence Address: VINSON & ELKINS, L.L.P., 1001 FANNIN STREET, 2300 FIRST CITY TOWER, HOUSTON, TX, 77002-6760, US

(73) Assignee: M & M INX CO.

(21) Appl. No.: 10/623,275

(22) Filed: Jul. 18, 2003

Related U.S. Application Data
(60) Provisional application No. 60/396,818, filed on Jul. 18, 2002.

(51) Int. Cl. A61K 7/42

(52) U.S. Cl. 424/59

(57) ABSTRACT

A topical composition for enhancement of a tattoo when applied to tattooed skin includes a UV protection agent and an optical brightener in a topical cosmetic carrier.

### CROSS REFERENCE TO RELATED APPLICATIONS

This Application claims benefit of U.S. Provisional Application Ser. No. 60/396,818, filed Jul. 18, 2002.

### FIELD OF THE INVENTION

The present invention relates to novel compositions of matter and methods for use of those compositions. More particularly, the invention relates to a topical cosmetic composition for use on tattooed skin. Most particularly, the invention relates to a topical cosmetic application useful in providing UV protection and optical enhancement to tattooed skin.

### BACKGROUND OF THE INVENTION

Common skin problems that may be associated with exposure to UV radiation include sunburn, premature aging as evidenced by wrinkling and loss of elasticity, and melanomas, among others. Tattooed skin is even more susceptible to these types of damage. Exposure of tattooed skin to UV radiation can additionally result in fading of the tattoo, loss of definition, loss of color, and overall dulling of the appearance of the tattoo. Individuals with tattoos, therefore, typically avoid prolonged sun exposure to the tattooed areas and often wear additional clothing to protect the tattoo from damage.

Sunscreen compositions are widely used to block or retard damage caused by UV radiation. Typical sunscreens include, but are not limited to creams, lotions, oils, sprays, sticks, wipes, or emulsions of oil-in-water or water-in-oil. Some preparations are “water-proof” and are more resistant to washing off the skin when a user swims or exercises. Formulations may also be tailored to dry or oily skin, sun-damaged skin, lips, and other special needs by methods known in the art. Sunscreens typically contain a sunscreen agent capable of blocking, absorbing, or reflecting UV radiation. These UV inhibitors, also known as light absorbers, light stabilizers, or light antioxidants, include both inorganic and organic compounds. Inorganic UV inhibitors include calamine, zinc oxide and titanium oxide, while organic compounds include hydroxyphenyl benzotriazoles, hindered amine light stabilizers, sterically hindered phenolic antioxidants, p-aminobenzoic acids, benzophenones, dioxybenzones, or salicylate esters. Many of these agents may be effective in reducing UV radiation exposure to the skin, but tattoos may still be damaged.

In addition to UV protection, therefore, it is also desirable to provide optical enhancement to the tattooed skin to increase the color clarity, definition, and overall brightness of the tattoo. Various optical brighteners are currently used in cosmetics to provide increased radiance and brightening of the skin or of the cosmetics of which they are a part. Typical optical brighteners include stilbene derivatives, coumarin derivatives, styryl derivatives, pyrazoline derivatives, naphthalamide derivatives or benzooxazole derivatives. Few of these brighteners, however, improve the color clarity, definition, or brightness of tattoos. Unfortunately, there is no composition formulated for topical application to tattooed skin that enhances the tattoos of the user while protecting the skin from UV damage.

### BRIEF SUMMARY OF THE INVENTION

The present invention addresses the deficiencies of the prior art by providing compositions formulated to enhance the color and clarity of tattoos and to protect the tattooed skin from UV radiation and damage. The present disclosure includes compositions that contain optical brighteners and UV inhibitors capable of providing these benefits to tattooed skin in a topical cosmetic application. In addition to UV protection and optical brightening, it is also desirable to provide therapeutic elements to aid in the healing of UV damaged tattooed skin while reducing the irritation caused by that damage. The present disclosure, therefore, also describes the optional use of therapeutic agents included in the topical cosmetic composition for application to tattooed skin.

Preferred compositions include at least one UV inhibitor and at least one optical brightener suitable for enhancing the color, clarity and brightness of tattooed skin in a cosmetically suitable carrier. Additionally, the cosmetic compositions may also have at least one therapeutic agent to aid in healing UV damaged tattooed skin and reducing the irritation caused by UV damage.

The present disclosure also includes methods of providing UV protection and visual enhancement of tattooed skin by applying a composition that includes a cosmetic carrier, at least one UV inhibitor and at least one optical brightener suitable for enhancing the color, clarity and brightness of tattooed skin. Additionally, the composition may also have at least one therapeutic agent to aid in healing UV damaged tattooed skin and reducing the irritation caused by UV damage.

### DETAILED DESCRIPTION OF THE INVENTION

Topical cosmetic and sunscreen compositions according to the present disclosure may be adapted to any type of acceptable vehicle or carrier. Many such carriers are known in the art and include, but are not limited to creams, lotions, oils, sprays, sticks, wipes, or emulsions of oil-in-water or water-in-oil. Some preparations are “water-proof” and are more resistant to washing off the skin when a user swims or exercises. Formulations may also be tailored to dry or oily skin, sun-damaged skin, lips, and other special needs by methods known in the art. The products designed for tattooed skin disclosed herein include at least one UV inhibiting agent and at least one optical brightener in any such carrier. Typical compositions include from about 0.1 to about 30%, or about 0.1 to 20% or about 0.1 to 10% by weight of a UV inhibitor and from about 0.1 to about 10% by weight of an optical brightener. It is understood in the art that a higher concentration of UV inhibitor results in a higher “SPF” for the composition, but that when the concentration is too high, it may become detrimental to the skin. The present disclosure, therefore, contemplates compositions that include any amount of UV inhibitor, from a minimal effective amount up to the highest concentration allowed by any federal or state agency that controls the formulation of such compositions. To improve shelf life of the compositions, at least 0.1-2% by weight of a preservative may be used. Additionally, if the composition is used as a therapeutic to aid in healing of UV damaged skin, it may include from about 0.1 to about 15% of a therapeutic agent.

One skilled in the art will recognize that a wide variety of cosmetic carriers are suitable for the disclosed compositions. Creams, lotions and ointments typically fall into one of four general classes: hydrocarbon bases, absorption bases, emulsion bases and water soluble bases. The type of carrier will depend, of course, on the method of application for a particular product, whether it be a cream, lotion, spray, stick, etc. Such carriers are well known in the art.

Ingredients that may be used in the disclosed compositions may include, but are not limited to, any of the following:

Moisturizers that function as a moisture barrier or to attract moisture from the environment:

cetyl alcohol (fatty alcohol);

dimethicone silicone—skin condition and anti-foam ingredient;

isopropyl lanolate, myristate, and palmitate;

lanolin and lanolin alcohols and oil;

octyl dodecanol;

oleic acid (olive oil);

stearic acid and stearyl alcohol;

Preservatives and antioxidants (including vitamins) that prevent product deterioration:

disodium, trisodium and tetrasodium salts of ethylenediamine tetraacetic acid (EDTA);

tocopherol (vitamin E);

Antimicrobials to fight bacteria:

butyl, propyl, ethyl, and methyl parabens;

DMDM hydantoin;

methylisothiazolinone;

phenoxyethanol;

quatemium-15;

Thickeners and waxes used in stick products:

candelilla, camauba, and microcrystalline waxes;

carbomer and polyethylene thickeners;

Solvents used to dilute:

butylene glycol and propylene glycol;

cyclomethicone (volatile silicone);

ethanol;

glycerin;

Emulsifiers:

glyceryl monostearate and distearates (also pearlescent agent);

lauramide DEA;

polysorbates;

pH Adjusters:

citric acid;

triethanolamine (TEA);

Film forming polymers:

C30-38 olefin/isopropyl maleate/MA copolymer;

PVP/eicosene copolymer;

Others:

magnesium aluminum silicate thickeners;

silica (silicon dioxide);

carbomers;

stearic acid;

talc (powdered magnesium silicate);

zinc stearate;

vitamins A, D, K (retinols, calciferols, and phylloquinones);

absorbents.

A moisturizing cream may contain, for example, talc, C12-15 alkyl benzoate, cyclopentasiloxane, glyceryl stearate and PEG-100 stearate, steareth-2, dimethicone, cetearyl alcohol, xanthum gum, allantoin and glycerin. Lotion carriers may include deionized water, xanthum gum, alantoin, glycerin, trisodium EDTA, isopropyl palmitate, cyclopentasiloxane, dimethicone, glyceryl stearate, PEG-100 stearate, steareth-2, cetearyl alcohol and octyldocecyl neopentanoate, for example. Water resistant creams may additionally include magnesium aluminum silicate, triethanolamine, polysorbate 20, and polysorbate 60, for example. A moisturizing cream carrier may include any of hexylene glycol, methyl gluceth-20, hydrogenated C6-14 polymers, glyceryl stearate and PEG-100 stearate, cetearyl alcohol, or it may include distearyldimonium chloride, cetyl alcohol, petrolatum, dimethicone, and glycerin. A preferred cosmetic carrier is an oil-in-water emulsion comprising 35-55% water, 1-5% polymeric emulsifier, 0.1-1% polymeric thickener, 1-3% glycerine, 1-20% dimethoxysilyldimethylamino-ethylaminopropyl silicone polymers, 1-5% PEG stearate, 1-5% caprylic capric triglyceride, 1-10% cyclomethicone, 0.1-2% triethylamine (TEA), and 1-10% of blended sodium hyaluronate and hydrolyzed glycosaminoglycans (Actiglide®, Active Organics, Lewisville, Tex., USA).

UV inhibitors are well known in the art. One skilled in the art will appreciate that a wide variety of UV inhibitors may be used to practice the present invention without undue experimentation. Preferred UV inhibitors include both organic and inorganic light absorbers, light stabilizers, or light antioxidants. More preferred UV inhibitors include calamine, zinc oxide, titanium oxide, hydroxyphenyl benzotriazoles, hindered amine light stabilizers, sterically hindered phenolic antioxidants, p-aminobenzoic acids, benzophenones, dioxybenzones, or salicylate esters. More preferred UV inhibitors include hydroxyphenyl benzotriazoles, hindered amine light stabilizers, or sterically hindered phenolic antioxidants. Most preferred UV inhibitors include branched and linear 2(2H-benzotriazole-2-yl)-6-(dodecyl)-4-methylphenol (sold under the trade name Tinuvin® 171 by CIBA Specialty Chemicals, Basel, Switzerland); mixed bis(1,2,2,6,6-pentamethyl-4-piperidinyl) sebacate (Tinuvin® 292, CIBA Specialty Chemicals); 2-(2′-hydroxy-3′,5′-di-tert-amylphenyl) benzotriazole (Tinuvin® 328, CIBA Specialty Chemicals); mixture of β-(3-(2H-benzotriazole-2-yl)-4-hydroxy-5-tert-butylphenyl) propionic acid, methyl ester and polyethylene glycol (PEG) 300 (Tinuvin® 1130, CIBA Specialty Chemicals); pentaerythritol tetrakis (3-(3,5-di-tert-butyl-4-hydroxyphenyl) proprionate) (Irganox® 1010, CIBA Specialty Chemicals); thiodiethylene bis[3-(3,5-di-tert-butyl-4-hydroxyphenyl) propionate] (Irganox® 1035, CIBA Specialty Chemicals); octadecyl-3-(3,5-di-tert-butyl-4-hydroxyphenyl) proprionate (Irganox® 1076, CIBA Specialty Chemicals); synergistic blend of pentaerythritol tetrakis (3-(3,5-di-tert-butyl-4-hydroxyphenyl) proprionate) (Irganox 1010) and Tris (2,4-di-tert-butylphenyl)phosphite (blend sold as Irganox® B 225, CIBA Specialty Chemicals); 2′,3-bis[[3-[3,5-di-tert-butyl-4-hydroxyphenyl]propionyl]]propionohydrazide (Irganox® MD 1024, CIBA Specialty Chemicals), ethylhexyl methoxycinnamate, or butyl methoxybenzolmethane.

Optical brighteners according to the present invention include, but are not limited to, stilbene derivatives, coumarin derivatives, styryl derivatives, pyrazoline derivatives, naphthalamide derivatives or benzooxazole derivatives. Preferred optical brighteners should enhance the color, clarity and brightness of tattooed skin when used according to the present invention, and may include, but are not limited to hexasodium-2,2′-[vinylenebis[3-sulfanato-4,1-phenylene)imino[6-diethylamino)-1,3,5-triazine-4,2-diyl]imino]]bis(benzene-1,4-disulphonate) (sold under the trade name Tinopal® SFP, CIBA Specialty Chemicals) and polyamide-12 triazaminostilbenedisulfonate.

One skilled in the art will appreciate the variety of therapeutic agents that may be suitable to aid in healing and reducing irritation in UV damaged skin. Preferred therapeutic agents comprise a combination of naturally occurring substances suitable to promote healing and reduce irritation in UV damaged skin. One preferred embodiment of such a therapeutic agent comprises 0.1-10% green tea concentrate, 0.1-5% glyoxyldiureide (Allantoin), 0.1-5% aloe barbadansis, 0.1-5% willowherb, 0.1-5% panthenol, and 0.1-10% tocopherol acetate.

Modern cosmetic compositions normally include preservatives to increase the shelf life of the composition. One skilled in the art will understand the various preservatives available and the suitability of various preservatives for different applications in relation to the cosmetic carrier being used. One preferred embodiment suitable for use with the present invention comprises a combination of 0.1-2% methyl and propyl paraben and 0.1-1% imidazolidinyl urea (Germall® 115, ISP Chemicals, Inc., Chatham, N.J., USA).

Other ingredients that may be included in the formulations include antihistamines, local anesthetics, antibiotics, antifungal agents, anti-inflammatory agents, water soluble steroid salts, or a combination of these. Others include buffering agents, chelating agents, stability enhancing agents, such as antioxidants, humectants (hydroscopic materials that prevent water loss from the preparation) such as glycerin, propylene glycol, and polyethylene glycols, gelling agents such as gelatin, PVP, PVA, cellulose derivatives (e.g., HPMC, HPC, HEC), acrylic derivatives (e.g., carboxymethyl cellulose, methyl cellulose and poly carbophil), natural and semi-synthetic polysaccharides (e.g., sodium alginate, chitosan, xanthan gum, acacia, carrageenan), inorganic gelling agents (veegum, bentonite), proteins (albumin, collagen), coloring agents, and aloe vera (as a thickening agent).

The following examples are included to demonstrate preferred embodiments of the invention. It should be appreciated by those of skill in the art that the compositions and methods of manufacture disclosed in the examples which follow represent examples discovered by and/or contemplated by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

| Phase | Ingredient | % |
|---|---|---|
| A | Purified water | 26.5 |
| | Pemulen TR-2¹ (2% soln) | 3.7 |
| | Carbopol 1382² (2% soln) | 7 |
| | Glycerine | 1.5 |
| | Methyl paraben | 0.3 |
| | Ethylhexyl methoxycinnamate | 7.5 |
| | Benzophenone-3 | 5 |
| | Ethylhexyl salicylate | 3 |
| | Butyl methoxydibenzoylmethane | 5 |
| B | Gransil FLD-55³ | 3 |
| | Gransil GCM⁴ | 2.5 |
| | Gransil PM gel⁵ | 2.5 |
| | GMS/PEG stearate | 1.5 |
| | Stearic acid | 1 |
| | Caprylic capric triglyceride | 2 |
| | Cyclomethicone | 3 |
| | Propyl paraben | 0.2 |
| C | Purified water | 2 |
| | TEA 99% | 0.5 |
| D | Purified water | 1.5 |
| | Germall 115⁷ | 0.3 |
| E | Purified water | 5 |
| | Actiglide J⁸ | 3.5 |
| | Tinopal SFP⁹ | 2 |
| F | Actiphyte green tea concentrate | 2 |
| | Allantoin | 2 |
| | Aloe barbadensis | 2 |
| | Willowherb | 1 |
| | Panthenol | 1 |
| | Tocopherol acetate | 2 |

¹Acrylates/C10-30 Alkyl Acrylate Crosspolymers (Noveon, Inc., Cleveland, OH)
²Noveon, Inc., Cleveland, OH
³Trifluoromethyl C1-4 Alkyl Dimethicone (Grant Industries, Inc., Elmwood Park, NJ)
⁴Cyclomethicone (and) Polysilicone - 11 (Grant Industries, Inc., Elmwood Park, NJ)
⁵Phenyl Trimethicone (and) Polysilicone - 11 (Grant Industries, Inc., Elmwood Park, NJ)
⁶Glycol Dibenzoate (and) PPG-15 Stearyl Ether Benzoate (Finetex, Inc., Elmwood Park, NJ)
⁷imidazolidinyl urea (ISP Chemicals, Inc., Chatham, NJ)
⁸blended sodium hyaluronate and hydrolyzed glycosaminoglycans (Active Organics, Lewisville, TX)
⁹hexasodium-2,2′-[vinylenebis[3-sulfanato-4,1-phenylene)imino[6-diethylamino)-1,3,5-triazine-4,2-diyl]imino]]bis(benzene-1,4-disulphonate) (CIBA Specialty Chemicals)

2 Combine phase B in a side kettle and heat to 80° C. while mixing;

3 Add phase B to phase A while homogenizing (use low sheer to prevent aeration);

4 Add phase C slowly while homogenizing and sweeping;

5 Mix at 80° C. for 20 minutes, then cool slowly to 45° C., discontinue homogenization continue sweeping;

6 At 45° C., at phase D and continue to cool;

7 Add phase E at 35° C. while sweeping (if counter-rotating sweep is not used, low hogenization in addition to sweep may be used);

8 Add phase F at 35° C. while sweeping.

### EXAMPLE 2

### Waterproof Formula I

| Phase | Ingredient | % |
|---|---|---|
| A | Isopropyl myristate | 4 |
| | Benzophenone-3 | 3 |
| | Ethylhexyl salicylate | 5 |
| | PPG-2 Myristyl ether propionate | 0.5 |
| | C12-15 alkyl benzoate | 4.5 |
| | Diethylhexyl naphthalate | 5 |
| B | Oxybenzone | 4 |
| | Stearyl alcohol | 0.3 |
| | Polyglyceryl-3 methyl glucose distearate | 3 |
| | C30-38 Olefin/isopropyl maleate/MA copolymer | 1 |
| | Disodium EDTA | 0.05 |
| C | Methyl paraben | 0.35 |
| | Propyl paraben | 0.35 |
| | Purified water | 45.6 |
| | Glycerine | 4 |
| | Butylene glycol | 2 |
| | Tinopal SFP | 2 |
| D | Carbomer | 0.2 |
| | Purified water | 5 |
| E | Willowherb | 1 |
| | Panthenol | 1 |
| | Tocopherol acetate | 2 |
| | Green tea | 2 |
| | Allantoin | 2 |
| | Aloe barbadensis | 2 |
| F | Triethanolamine | 0.15 |

1 Combine the ingredients for phase A in a jacketed kettle with homogenizer and side-sweep, heat to 80° C.;

2 Increase heat to 90° C. add phase B with continuous stirring;

3 Heat water to 85° C., preblend preservatives and glycerine, add to water;

4 Add phase C slowly while homogenizing and sweeping;

5 Predisperse the carbomer in water, add phase D;

6 Mix at 89° C. for 20 minutes, and then cool slowly to 45° C., discontinue homogenization but continue sweeping;

7 At 45° C. add phase D and continue to cool;

8 Add phase F at 35° C. while sweeping.

### EXAMPLE 3

### Waterproof Formula II

| Phase | Ingredient | % |
|---|---|---|
| A | Purified water | 34.65 |
| | Pemulen (2% solution) | 15 |
| | Propylene glycol | 1 |
| | Disoduim EDTA | 0.1 |
| | Methyl paraben | 0.35 |
| | Propyl paraben | 0.35 |
| | Tinopal SPF | 2 |
| B | Oxybenzone | 4 |
| | Ethylhexyl methoxycinnamate | 7.5 |
| | Ethylhexyl salicylate | 5 |
| | Phenyltrimethicone | 3 |
| | Iso-eicosene | 2 |
| | Finsolv TPP | 4 |
| | Cetyl phosphate | 0.2 |
| | C30-38 Olefin/isopropyl maleate/MA copolymer | 4 |
| C | TiO₂ paste (50%) | 4 |
| D | Cyclomethicone | 1 |
| E | Aloe Barbadensis | 2 |
| | Willowherb | 1 |
| | Panthenol | 1 |
| | Tocopherol Acetate | 2 |
| | Green tea | 2 |
| | Allantoin | 2 |
| F | Purified water | 1 |
| | Triethanolamine | 0.85 |

1 Combine the ingredients for phase A in a jacketed kettle with homogenizer and side-sweep, heat to 90° C.;

2 Separately heat phase B to 90° C.;

3 Add phase C to phase B and homogenize until fully dispersed;

4 Add phase A and continue to homogenize for 15 minutes;

5 Remove heat and cool to 75° C., add phases D and E under homogenization;

6 Cool to 60° C., add phase F, cool to 25° C.

What is claimed is:

1. A composition formulated for application to tattooed skin, comprising an optical brightener in an amount effective to enhance the color of a tattoo on said skin.

2. The composition of claim 1, further defined as comprising at least 0.1-10% by weight of an optical brightener.

3. The composition of claim 1, further comprising about 0.1-30% by weight of a UV inhibitor.

4. The composition of claim 1, further comprising about 0.1-20% by weight of a UV inhibitor.

5. The composition of claim 1, further comprising about 0.1-10% by weight of a UV inhibitor.

6. The composition of claims 3, 4 or 5 wherein the UV inhibitor is selected from the group consisting of ethylhexyl methoxycinnamate, benzophenone-3, ethylhexyl salicylate, butyl methoxydibenzoylmethane, or homosalate.

7. The composition of claim 1 wherein the optical brightener is hexasodium-2,2′-[vinylenebis[3-sulfanato-4,1-phenylene)imino[6-diethylamino)-1,3,5-triazine-4,2-diyl]imino]]bis(benzene-1,4-disulphonate).

8. The composition of claim 1 formulated as a lotion, cream, spray, oil, stick, oil-in water emulsion or water-in-oil emulsion.

9. The composition of claim 1, further comprising at least 0.1-2% by weight of a preservative.

10. The composition of claim 9, wherein said preservative is 0.1-2% methyl and propyl paraben, 0.1-1% imidazolidinyl urea, or a combination thereof.

11. The composition of claim 1, further comprising at least 0.1-15% of a therapeutic agent.

12. The composition of claim 11, wherein the therapeutic agent is 0.1-10% green tea concentrate, 0.1-5% glyoxyldiureide (Allantoin), 0.1-5% aloe barbadansis, 0.1-5% willowherb, 0.1-5% panthenol, 0.1-10% tocopherol acetate, or a combination thereof.

13. The composition of claim 1, further comprising an antihistamine, a local anesthetic, an antibiotic, an antifingal agent, an anti-inflammatory agent, a water soluble steroid salt, or a combination of these.

14. The composition of claim 1, further comprising a buffering agent, a chelating agent, an antioxidant, a humectant, or a gelling agent.

15. An ultra violet light protection cream composition for protecting and enhancing the optical appearance of tattoos which in combination comprises by weight:
a) an oil in water emulsion comprising about 30-55% water, about 5% polymeric emulsifier, about 0.2% polymeric thickener, about 1.5% glycerine, about 10% dimethoxysilyldimethylamino-ethylaminopropyl silicone polymers, about 1.5% PEG stearate, about 2% caprylic capric triglyceride, about 3% cyclomethicone, about 0.5% triethylamine, and about 3.5% blended sodium hyaluronate and hydrolyzed glycosaminoglycans;
b) a therapeutic additive package comprising about 2% green tea concentrate, about 2% allantoin, about 2% aloe barbadansis, about 1% willowherb, about 1% panthenol, and about 2% tocopherol acetate;
c) a preservative combination of methyl & propyl paraben at about 0.5% and about 0.3% imidazolidinyl urea;
d) UV inhibitors comprising ethylhexyl methoxycinnamate, benzophenone-3, ethylhexyl salicylate, butyl methoxydibenzoylmethane, homosalate, or a combination thereof, which are present in the proportion of about 5-10% and are in the emulsion mixture; and
e) hexasodium-2,2′-[vinylenebis[3-sulfanato4,1-phenylene)imino[6-diethylamino)-1,3,5-triazine-4,2-diyl]imino]]bis(benzene-1,4-disulphonate), present in the proportion of about 0.05-4.0%.

16. A method of providing UV-protection and optical enhancement to tattooed skin comprising applying a topical cosmetic composition to said tattooed skin, said composition comprising at least 0.1-20% by weight of a UV inhibitor and at least 0.1-10% by weight of an optical brightener.

17. The method of claim 16 wherein the UV inhibitor is selected from the group consisting of ethylhexyl methoxycinnamate, benzophenone-3, ethylhexyl salicylate, butyl methoxydibenzoylmethane, or homosalate.

18. The method of claim 16 wherein the optical brightener is hexasodium-2,2′-[vinylenebis[3-sulfanato-4,1-phenylene)imino[6-diethylamino)-1,3,5-triazine-4,2-diyl]imino]]bis(benzene-1,4-disulphonate).

19. An improved sun-protection cosmetic for topical administration, wherein the improvement comprises an optical brightener in an amount effective to enhance the color, clarity or brightness of a tattoo when applied to tattooed skin.

20. The improved sun-protection cosmetic of claim 19, wherein the optical brightener is hexasodium-2,2′-[vinylenebis[3-sulfanato-4,1-phenylene)imino[6-diethylamino)-1,3,5-triazine-4,2-diyl]imino]]bis(benzene-1,4-disulphonate).

21. A composition for topical application comprising, by weight:
about 35% purified water; about 10.7% of a 2% solution of polymeric thickeners;
about 1.5% glycerine; about 0.3% methyl paraben; about 7.5% ethylhexyl methoxycinnamate; about 5% benzophenone-3; about 3% ethylhexyl salicylate;
about 5% butyl methoxydibenzoylmethane; about 8% silicones and di and trimethicones; about 1.5% GMS/PEG stearate; about 1% stearic acid; about 2% caprylic capric triglyceride; about 3% cyclomethicone; about 0.2% propyl paraben;
about 0.5% triethylamine 99%; about 0.3% imidazolidinyl urea; about 3.5% blended sodium hyaluronate and hydrolyzed glycosaminoglycans; about 2% hexasodium-2,2′-[vinylenebis[3-sulfanato-4,1-phenylene)imino[6-diethylamino)-1,3,5-triazine-4,2-diyl]imino]]bis(benzene-1,4-disulphonate; about 2% actiphyte green tea concentrate; about 2% allantoin; about 2% aloe barbadensis; about 1% willowherb;
about 1% panthenol; and about 2% tocopherol acetate.

22. A composition for topical administration comprising by weight:
about 50.6% purified water; about 4% isopropyl myristate; about 3% benzophenone-3; about 5% ethylhexyl salicylate; about 0.5% PPG-2 myristyl ether propionate;
about 4.5% C12-15 alkyl benzoate; about 5% diethylhexyl naphthalate; about 4% oxybenzone; about 0.3% stearyl alcohol; about 3% polyglyceryl-3 methyl glucose distearate; about 1% C30-38 olefin/isopropyl maleate/MA copolymer; about 0.05% disodium EDTA; about 0.35% methyl paraben; about 0.35% propyl paraben; about 4% glycerine; about 2% butylene glycol; about 2% hexasodium-2,2′-[vinylenebis[3-sulfanato-4,1-phenylene)imino[6-diethylamino)-1,3,5-triazine-4,2-diyl]imino]]bis(benzene-1,4-disulphonate; about 0.2% carbomer; about 1% willowherb, about 1% panthenol; about 2% tocopherol acetate; about 2% green tea;
about 2% allantoin; about 2% aloe barbadensis; and about 0.15% triethanolamine.

23. A composition for topical administration comprising by weight:
about 35.65% purified water; about 15% polymer; about 1% propylene glycol; about 0.1% disoduim EDTA; about 0.35% methyl paraben; about 0.35% propyl paraben; about 2% hexasodium-2,2′-[vinylenebis[3-sulfanato-4,1-phenylene)imino[6-diethylamino)-1,3,5-triazine-4,2-diyl]imino]]bis(benzene-1,4-disulphonate; about 4% oxybenzone; about 7.5% ethylhexyl methoxycinnamate; about 5% ethylhexyl salicylate; about 3% phenyltrimethicone; about 2% iso-eicosene; about 4% Finsolv TPP; about 0.2% cetyl phosphate; about 4% C30-38 Olefin/isopropyl maleate/MA copolymer; about 4% TiO₂ paste (50%); about 1% cyclomethicone; about 2% aloe barbadensis; about 1% willowherb; about 1% panthenol; about 2% tocopherol acetate; about 2% green tea; about 2% allantoin; and about 0.85% triethanolamine.
